(19) United States
(12) Patent Application Publication
(10) Pub. No.: US 2021/0161643 A1
(43) Pub. Date: Jun. 3, 2021

(54) METHODS AND DEVICES FOR THE TREATMENT OF PULMONARY DISORDERS WITH IMPLANTABLE VALVES

(71) Applicant: EOLO MEDICAL INC., New York, NY (US)

(72) Inventors: Sean TOTTEN, New York, NY (US); Jason LEE, New York, NY (US); Lilip LAU, New York, NY (US); Don TANAKA, New York, NY (US); Mark GELFAND, New York, NY (US)

(21) Appl. No.: 17/263,034

(22) PCT Filed: Jul. 22, 2019
(86) PCT No.: PCT/US2019/042794

§ 371 Date: Jan. 25, 2021

Related U.S. Application Data
(60) Provisional application No. 62/702,048, filed on Jul. 23, 2018.
(60) Provisional application No. 62/726,726, filed on Sep. 4, 2018.
(60) Provisional application No. 62/747,528, filed on Oct. 18, 2018.

(30) Foreign Application Priority Data
Oct. 18, 2018 (CN) 201811213823.7

(51) Int. Cl. A61F 2/04; A61B 17/12; A61F 2/966

(52) U.S. Cl. CPC A61F 2/04; A61B 17/12104; A61F 2/966; A61F 2002/9528; A61B 2017/12054; A61F 2220/0016; A61F 2002/043

(57) ABSTRACT

A flow control device (241, 260, 300, 350, 450, 480, 500) for a bronchial passageway including: a one-way valve (273, 313, 360, 478, 511); a hollow structural frame (242, 302, 352, 453, 468, 509) housing the one-way valve, wherein the structural frame is expandable from a collapsed configuration to an expanded configuration; and a sealing membrane (316, 470, 512) mounted to at least a distal portion of the structural frame, wherein the sealing membrane forms an enclosed wall defining at least a portion of an airflow passage through the flow control device, and the one-way valve is included in the airflow passage.

### TECHNICAL FIELD

The field of the invention is lung volume reduction devices used to treat hyper-inflated lung, for example in patients diagnosed with chronic obstructive pulmonary disease (COPD), emphysema, asthma, bronchitis. The invention relates to lung volume reduction devices such as deployable valves configured to be delivered through the airway to the lung with minimally invasive techniques.

### BACKGROUND

Hyper-inflated lung is a lung disease that makes it hard to breathe. COPD is a major cause of disability and is the third leading cause of death in the United States. The symptoms and effects of COPD often worsen over time, such as over years, and can limit the ability of a person suffering from COPD to do routine activities. Current medical techniques offer no solution for reversing the damage to the airways and lungs associated with COPD.

COPD often does not affect all air sacs or alveoli equally in a lung. A lung may have diseased regions in which the air sacs are damaged and unsuited for gas exchange. The same lung may have healthy regions (or at least relatively healthy regions) in which the air sacs continue to perform effective gas exchange. The diseased regions may be large, such as 20 to 30 percent or more of the lung volume.

The diseased regions of the lung occupy volume in the pulmonary cavity, which could otherwise be occupied by the healthy portion of the lung. If the healthy regions(s) of the lung were allowed to expand into the volume occupied by the diseased regions, the healthy regions could expand and fill with air to allow the air sacs in the healthy region to exchange oxygen for carbon dioxide.

In U.S. Patent Application Publication 2014/0058433 describes methods and devices are adapted for regulating fluid flow to and from a region of a patient's lung, such as to achieve a desired fluid flow dynamic to a lung region during respiration and/or to induce collapse in one or more lung regions. Pursuant to an exemplary procedure, an identified region of the lung is targeted for treatment. The targeted lung region is then bronchially isolated to regulate airflow into and/or out of the targeted lung region through one or more bronchial passageways that feed air to the targeted lung region.

U.S. Pat. No. 7,842,061 discloses an intra-bronchial device placed and anchored in an air passageway of a patient to collapse a lung portion associated with the air passageway. The device includes a support structure, an obstructing member carried by the support structure that reduces ventilation to the lung portion by preventing air from being inhaled into the lung portion, and at least one anchor carried by the support structure that anchors the obstruction device within the air passageway. The anchor may engage the air passageway wall by piercing or friction, include a stop dimensioned for limiting the piercing of the air passageway wall, and may be releasable from the air passageway for removal of the intra-bronchial device. The anchors may be carried by a peripheral portion of the support structure, or by a central portion of the support structure. The obstructing member may be a one-way valve.

WO International Publication Number 2004010845 discloses a flow control device for a bronchial passageway. The device can include a valve member that regulates fluid flow through the flow control device, a frame coupled to the valve member, and a membrane attached to the frame. At least a portion of the flow control device forms a seal with the interior wall of the bronchial passageway when the flow control device is implanted in the bronchial passageway. The membrane forms a fluid pathway from the seal into the valve member to direct fluid flowing through the bronchial passageway into the valve member.

However, there remains a need for a lung volume reduction device and procedure that effectively treats patients suffering from a hyperinflated lung that is also affordable, quick to implant, easily assessable and removable, and safe.

### SUMMARY

This disclosure is related to methods, devices, and systems for reducing volume of a hyper-inflated lung, for example in a patient suffering from COPD.

One aspect of the disclosure is a device for reducing volume of a patient's diseased lung lobe comprising a proximal end, a distal end, a deployable structural frame, a sealing element, a valve, and a retention element. The device may be embodied as an endobronchial valve, such as a lobar one-way valve. These functions may be served by distinct structures or in some embodiments one or more structures may provide one or more of these functions.

The structural frame for the endobronchial valve may be made from a laser cut Nitinol tube and comprises struts in a straight, spiral or offset configuration that are connected to a tubular segment at the proximal end and a tubular segment at the distal end. The Nitinol tube may be superelastic Nitinol and have an outer diameter (OD) of 0.083″ (2.1 mm), and an internal diameter (ID) of 0.072″ (1.8 mm). The deployable structural frame may be deployable from a contracted state to an expanded state and wherein the ratio of the diameter in the expanded state to the diameter in the contracted state is in a range of 3 to 6 (e.g., 5 to 6). The Nitinol structural frame may be shape set so that the struts have radially extending proximal and distal sections connected by a central section that is substantially parallel to the axis of the device. The central section may have a length in a range of 0.13″ to 0.19″ (3.3 mm to 4.8 mm). The structural frame may be made from a bioabsorbable material.

The structural frame may further comprise a coupler on its proximal end. The coupler may be configured to mate with a delivery tool and transmit torque and translation applied to the delivery tool to the device.

The endobronchial valve, such as a one way lobar valve, may have a sealing element that is a flexible membrane connected to the structural frame.

The endobronchial valve may include a one-way valve that permits air to flow in a direction from the distal end to the proximal end.

Also disclosed herein is a method of treating a patient with COPD comprising delivering a lobar valve through a working channel of a bronchoscope and deploying the lobar valve in a lobar bronchus that feeds a diseased lobe of the patient's lungs so that the lobar valve permits air to be released from the diseased lobe and air is not permitted to pass into the diseased lobe. The method may further comprise affixing a retention element of the lobar valve to an airway carina distal to the lobar bronchus. The retention element may be an airway carina screw or an airway carina clip. The valve may be positioned in the lobar bronchus such that the axis of the valve is not parallel with the axis of the lobar bronchus.

### BRIEF DESCRIPTION OF THE DRAWINGS

FIG. 1 is a schematic illustration of a patient's lungs and airways with the right middle lobe omitted.

FIG. 2 is a schematic illustration of an embodiment of a structural frame.

FIG. 3A is a schematic illustration of an embodiment of a lobar valve.

FIG. 3B is a schematic illustration of another embodiment of a lobar valve.

FIG. 4A is a schematic illustration of another embodiment of a lobar valve.

FIG. 4B is a schematic illustration of another embodiment of a lobar valve.

FIG. 4C is a schematic illustration of another embodiment of a lobar valve.

FIG. 4D is a schematic illustration of cross section of the embodiment of FIG. 4C.

FIG. 5A is a schematic illustration of another embodiment of a lobar valve.

FIG. 5B is a schematic illustration of side view of the embodiment of FIG. 5A.

FIG. 5C is a schematic illustration of cross section of the embodiment of FIG. 5A.

FIG. 6A is a schematic illustration of a delivery tool delivering a valve device through a working channel of a bronchoscope.

FIG. 6B is a schematic illustration of a delivery tool holding an implantable valve within a delivery sheath in a contracted delivery state.

FIG. 6C is a schematic illustration of a delivery tool advancing an implantable valve from a delivery sheath.

FIG. 6D and 6E are schematic illustrations showing alternative embodiments of delivery tools.

FIG. 6F is a schematic illustration of a delivery tool advancing an implantable valve from a delivery sheath with a forceps tool grasping an airway bifurcation carina.

FIG. 7A is a schematic illustration a delivery sheath holding a lobar valve connected to a delivery tool with the rest of the lobar valve advanced from the delivery sheath in its expanded unconstrained state.

FIG. 7B is a schematic illustration of a lobar valve connected to a delivery shaft and contained in a delivery sheath that is advanced through a working channel of a bronchoscope positioned in a target lobar bronchus.

FIG. 7C is a schematic illustration of a lobar valve connected to a delivery shaft and partially advanced from a delivery sheath (stage 1) that is advanced through a working channel of a bronchoscope positioned in a target lobar bronchus.

FIG. 7D is a schematic illustration of a lobar valve fully advanced from a delivery sheath (stage 2) and released from a delivery shaft and positioned in a target lobar bronchus.

### DETAILED DESCRIPTION

The disclosure herein is related to systems, devices, and methods for modifying air flow to and from a portion of a patient's lung with an implantable device, which may be substantially diseased in order to reduce the volume of trapped air in the targeted portion of lung, thereby increasing the elastic recoil of the remaining lung volume.

The inventors conceived of and disclose herein, implantable lung volume reducing devices and medical techniques for implanting lung volume reduction devices through the trachea and bronchi, using minimally-invasive deployment, bronchoscopic and surgical techniques. The device may be embodied as an endobronchial valve, such as a one-way lobar valve.

The invention may be embodied as a novel treatment for patients suffering from hyper-inflated lung (e.g., emphysema, COPD, bronchitis, asthma) comprising the application of a minimally invasive bronchoscopy technique to implant a lung volume reduction device into a lung airway of a patient. The implantable lung volume reduction devices, which may be generally referred to as a “lobar valves” disclosed herein are intended to be placed in an airway trunk of a lobe such that the single valve regulates air flow to or from the complete lobe, which may have benefits over previously attempted valves that were intended for multiple valve placement in higher generation airways. Benefits of a lobar valve may include lower cost, faster procedure, easier implantation, easier removal, and stronger retention. However, some features of devices disclosed herein may be novel and useful for use in higher generation airways and are not limited to devices configured for placement in a trunk of a lobe.

Anatomy and Design Inputs and Challenges:

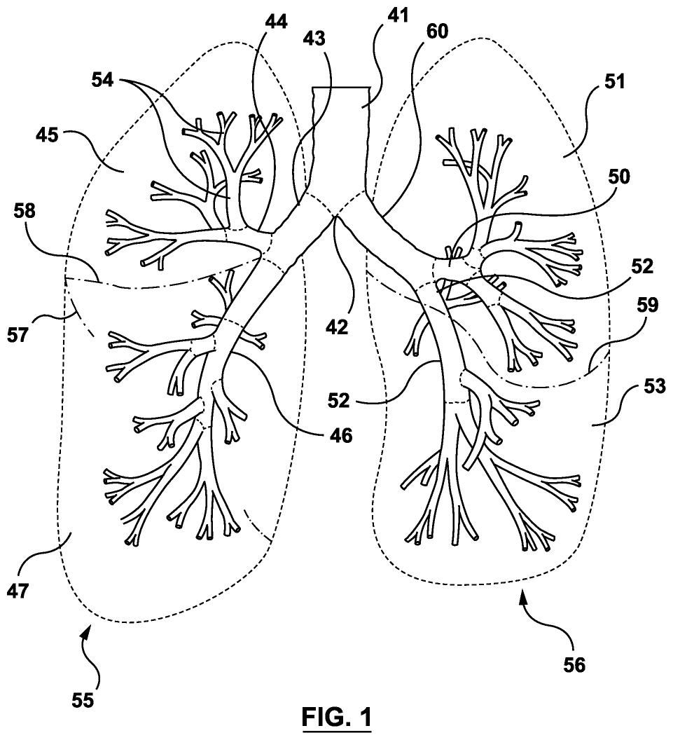

FIG. 1 is a schematic illustration of some anatomical features of the lungs. Air passes through the trachea 41, which divides 42 into the right and left main or primary bronchi 43 and 60. The lungs normally have clear anatomical divisions known as lobes. The right lung 55 is divided into three lobes called superior 45, middle (not shown for simplicity) and inferior 47 lobes, by the oblique 57 and horizontal 58 fissures that are folds of the visceral pleura. The left lung 56, which is slightly smaller, is divided into a superior 51 and inferior 53 lobe, by an oblique fissure 59. The term “proximal direction” refers to the direction along an airway path that points toward the patient's mouth or nose and away from the patient's lungs. In other words, the proximal direction is generally the same as the expiration direction when the patient breathes. The term “proximal section” or “proximal end” of a device implanted in a patient's airway refers to the section or end of the device intended to face the proximal direction. The term “distal direction” refers to the direction along an airway path that points toward the patient's lung and away from the mouth or nose. The distal direction is generally the same as the inhalation or inspiratory direction when the patient breathes. The term “distal section” or “distal end” of a device implanted in a patient's airway refers to the section or end of the device intended to face the distal direction.

Lobar valves 241, 300, 260, 350, 450, 480, 500 may be implanted in a secondary bronchus, also known as a lobar bronchus. Humans have one lobar bronchus providing air passage to each lobe of the lung, including three in the right lung and two in the left lung. The right side lobar bronchi include the right upper lobar bronchus 44, right middle lobar bronchus (not shown for simplicity), and right lower lobar bronchus 46. The left side lobar bronchi include the left upper lobar bronchus 50 and left lower lobar bronchus 52. Overlapping cartilage plates of the lobar bronchi provide structural strength to maintain patency of these bronchi. The average diameter of human lobar bronchi is about 8.3 mm and the average length is about 19 mm (e.g., in a range of about 15 to 30 mm).

Design considerations of the lobar valve embodiments disclosed herein include delivery, ease of use and cost.

The lobar valve may be delivered through a working channel of a bronchoscope. The lobar valve and delivery tools may be sized to pass freely through a working channel of a bronchoscope. For example, a lobar valve adapted to be delivered with a delivery tool through a working channel with a 2.8 mm lumen may have a maximum diameter of 2.6 mm (e.g., a maximum diameter of 2.5, 2.4, 2.3, 2.2. 2.1 mm). In some embodiments lobar valves may comprise a structural frame having a delivery state and deployed state, wherein the delivery state has a maximum diameter in a range of 2 (0.0787″) to 2.5 mm (0.0984″), preferably 2.11 mm (0.083″), and in the unrestricted deployed state has a maximum diameter in a range of 10.16 mm (0.4″) to 14 mm (0.551″), preferably about 12.42 mm (0.489″), which may be configured for placement in a lobar bronchus having an average diameter in a range of about 7 to 12 mm. For example, the ratio of the maximum outer diameter of the unconstrained state to the maximum diameter of the constrained delivery state may be in a range of 4:1 to 7:1, for example about 5.45:1. Due to the relatively larger diameter and short length of lobar bronchi, lobar valves may have a smaller length to diameter ratio in an expanded unconstrained state than current devices intended for more distal positioning. For example, a lobar valve may have a length in a range of 4 mm to 6 mm in its unconstrained state and a length to diameter ratio in a range of 0.545 to 0.286. Lobar valves may be provided having various sizes to be used with various airway diameters and generally have a structural frame having an unrestricted deployed state with a maximum diameter in a range of 5 to 30% (e.g., about 20%) greater than the diameter of the target airway. However, a feature of implantable valve designs disclosed herein that improves speed and ease of delivery includes an ability to fit and function properly in a wide range of airway diameters (e.g., 7 to 12 mm), lengths (e.g., 5 to 15 mm), and geometries (e.g., circular, oval, or irregular). The target airway may be measured using CT or other medical imaging or with a sizing device delivered through a bronchoscope. A membrane may be connected to a structural frame to function as an airway seal or an air flow control valve. The structural frame along with the connected membrane(s) in the delivery state may have a maximum diameter less than 2.7 mm (e.g., less than 2.6, 2.5, 2.4, 2.3, 2.2, 2.1 mm), preferably a maximum diameter of about 2.3 mm. Alternative embodiments of lobar valves may have different dimensions to allow them to be delivered through bronchoscope working channels having different diameters. Optionally, lobar valves in an unconstrained state may have a noncircular cross-section (e.g., ovoid, oval, irregular), which may have an improved fit in a bronchus having a noncircular cross-section. Alternatively, a lobar valve may be adapted to conform to a noncircular airway cross-section or irregular airway wall surface.

Ease of use and procedural expediency is a desired requirement. The lobar valve may be designed to be consistently delivered to a correct location with average physician skill. Compared to valves that are implanted at higher generation airways implanting a lobar valve may be a faster procedure because only one valve needs to be implanted to affect an entire lobe, the lobar bronchi are larger, more proximal and hence easier to access and find than distal higher generation bronchi. Also, assessing the function of a single implanted lobar valve is faster and easier compared to assessing multiple distally implanted valves.

A lobar valve and procedure for implanting one may cost less compared to implanting multiple higher generation valves in particular since there is only one device to implant and the procedure is faster.

Design considerations may also consider particular challenges for placement in a lobar bronchus. For example, the length of a lobar bronchi is relatively short, the length to diameter ratio is considerably smaller, the cross section of a lobar bronchus is radially asymmetrical (e.g., ovular or irregular), and the diameter of the lumen is inconsistent along the length of the lobar bronchus (e.g., flared at the proximal, distal or both ends). Furthermore each particular lobar bronchus in a patient has unique characteristics such as the angle of approach and geometry.

Lobar valves 241 may comprise a structural frame that is deployable from a contracted delivery state to an expanded deployed state, a sealing membrane, a one-way valve, and a retention element. These elements may be mixed and matched and embodiments are not limited to the combination of these elements presented in the figures.

Structural Frames:

Lobar valves 241 have a deployable structural frame 242 that may be made from a laser cut round tube, for example made from a biocompatible material such as superelastic Nitinol (e.g., a tube having an OD of 0.083″ and ID of 0.072″, a tube having an OD in a range of 0.070″ to 0.085″ and a wall thickness in a range of 0.005″ to 0.015″). A structural frame 242 may have a series of interconnected struts providing the ability of the frame to be flexible, to expand and contract from a contracted delivery state to an expanded deployed state, and to provide support for a sealing membrane, valve and retention element. In its contracted delivery state, the structural frame may have a diameter approximately the diameter of the laser cut tube it is cut from. A Nitinol structural frame may be laser cut from a Nitinol tube and shape set to its unconstrained expanded state. An alternative method of manufacture may include a structural frame made from shape set Nitinol wire. Shape setting a Nitinol, or other shape memory material, frame in its unconstrained expanded state allows the structure to elastically deform toward its shape set applying an elastic force when constrained by a target airway having a diameter smaller than the diameter of the frame in its fully expanded state. For example, the diameter of the frame in its fully expanded state may be in a range of 5% to 80% (e.g., about 10% to 30%) greater than the diameter of the target airway. This also allows the device to be contracted to its delivery state when loaded and contained in a delivery sheath and deployed to its expanded state when advanced out of the delivery sheath. The structural frame may have a proximal end and a distal end wherein the proximal end may comprise a coupler that mates with a delivery device and may have a notch that allows the coupler to transmit rotational and translational force from the delivery element to the structural frame. The coupler may be used as a graspable protrusion to grasp with a bronchoscopic tool to manipulate the device during implantation, repositioning, or removal.

In an embodiment a lobar valve having an unconstrained deployed state with a maximum diameter of 12 mm (0.472″) may be adapted for placement and to create a sufficient air seal in a range of airway sizes between about 6 mm (0.236″) to 10 mm (0.394″) and a larger version having a maximum diameter of 14 mm (0.551″) may be adapted for placement and to create a sufficient air seal in a range of airway sizes between about 7 mm (0.276″) to 12 mm (0.472″). Furthermore, once placed the structural frame may expand and contract with movement of the bronchus (e.g., during elastic recoil). The shape of the structural frame or use of its retention element may be resistant to tilting or may function properly when positioned in a range of angles with respect to the axis of the bronchus. Also, the structural frame may be compressed after it has been fully deployed allowing for repositioning. For example, a structural frame may be compressed by grasping or coupling a delivery tool to the frame's coupler and at least partially withdrawing it into a delivery sheath.

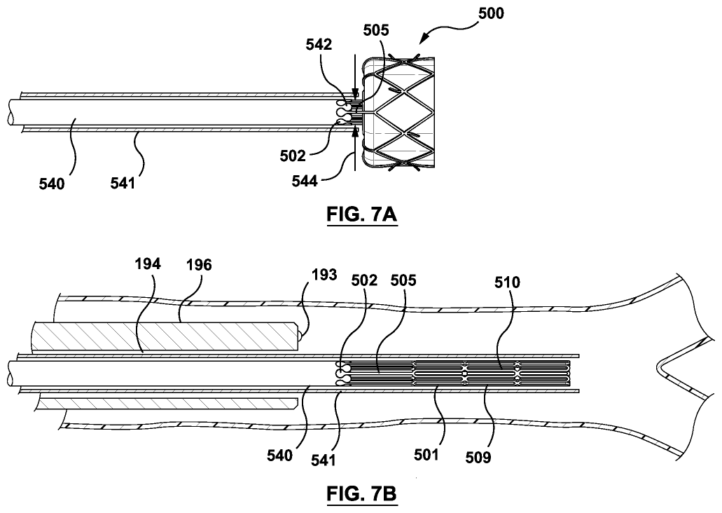

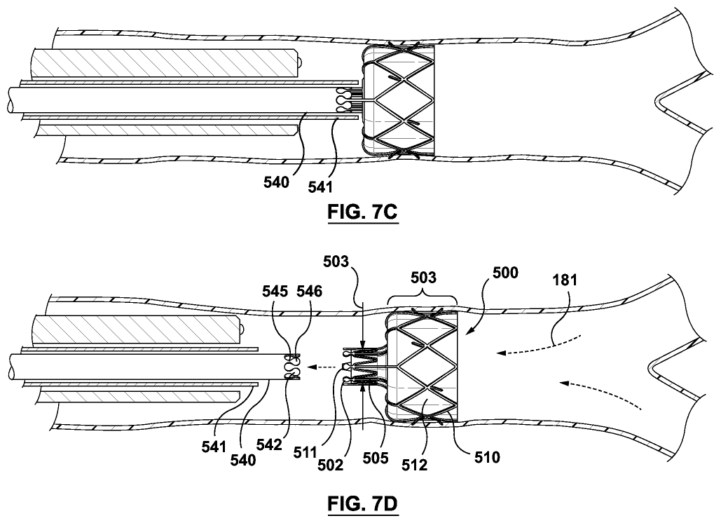

In its contracted delivery state, for example as shown in FIG. 7B, a structural frame 509 including it's interconnected struts 510, spokes 501, valve housing 505 and coupler 502 may be sufficiently flexible to pass through a lumen 194 of an endoscope 196 (e.g., bronchoscope) when the endoscope is bent to traverse a tortuous airway (e.g., having a radius of curvature as small as 15 mm).

Optionally or alternatively, a structural frame may be made from a bioresorbable material such as a laser cut polymer matrix (e.g., PLA, PLAGA, PDLLA) tube.

Optionally or alternatively, a structural frame may be balloon expandable or made from a plastically deformable material such as plastic, cobalt chrome alloy, martensitic Nitinol, stainless steel, silicone or urethane.

Optionally or alternatively, a structural frame may be impregnated with an agent such as an antifungal, antibacterial, antimitotic, or anti-inflammatory agent that may improve patient response to implanting the device.

Optionally, a coupler may be laser cut from the same tube as the structural frame or be connected (e.g., welded, bonded) to the structural frame. A delivery/removal tool may be a custom designed device made to mate with and apply rotational or translational forces to the valve. Alternatively, a delivery/removal tool may be a standard forceps catheter for use in a bronchoscope working channel.

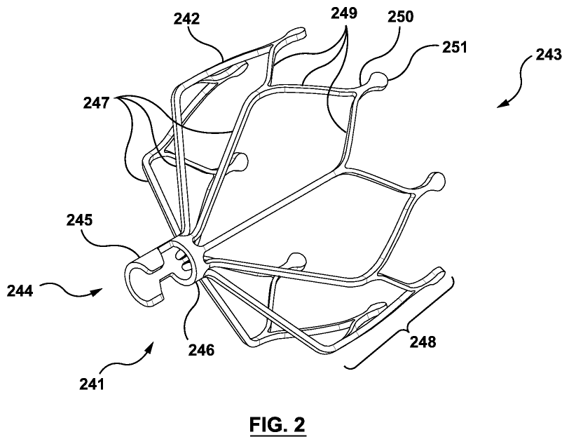

In some embodiments of lobar valves, for example as shown in FIGS. 2, 3A, 3B, 4A, 4B, 4C, and 5A, structural frames may have interconnected struts forming an expandable wall contact region 248, 309, 454, wherein the proximal end of the wall contact region is connected to spokes 247, 307, 354, that are connected to an optional valve housing and a coupler 245, 304, 356.

In these embodiments, the wall contact region may be adapted to comply to lobar bronchi that have oval or irregular lumen cross sections; the device may comply to irregular airway surfaces creating a seal on surfaces having bumps, ridges, grooves or other non-smooth surface; the device may have an overall length that is suited for fitting in lobar bronchi; the valve may be more easily coughed out of the body if it becomes dislodged from its implanted position; the device may be suitable for implanting in a wider range of lobar bronchi sizes and shapes.

Wall Contact Region

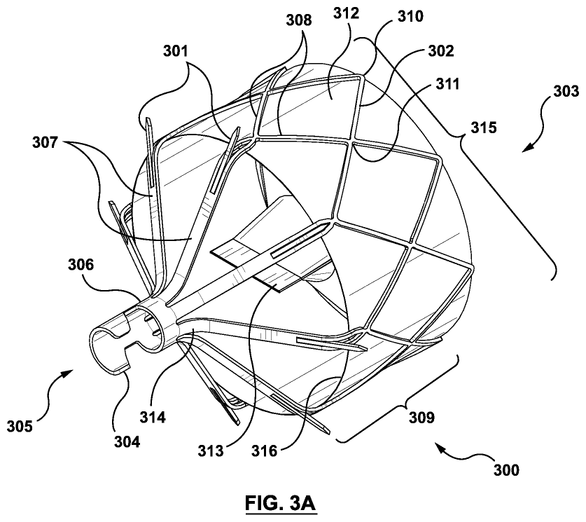

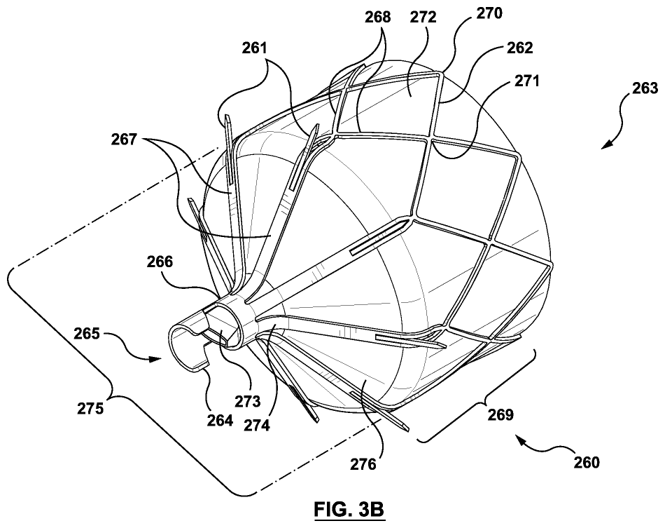

Referring to FIG. 3A as an example, an implantable lobar valve 300 shown in its unconstrained expanded state, has a wall contact region 309 toward the distal end 303 of the device 300 formed by a structural frame 302 with a sealing membrane 312 affixed to the structural frame. The wall contact region has a somewhat cylindrical shape that functions to press against the inner wall of a target airway (e.g., lobar bronchus) restricting or impeding airflow between the wall contact region of the device and the airway wall and to hold a one-way valve 313 in a lumen of the airway so air flow through the airway is significantly limited to passing through the valve. The wall contact area 309 provides an outward contact force and friction to the airway wall to contribute to resisting longitudinal movement in the airway so it remains where it is implanted. The wall contact area 309 may have flexibility and elasticity to conform to non-cylindrical (e.g., irregular, oval, tapered, flared) or non-smooth (e.g., bumpy, ridged, contoured) airways or alternatively apply a greater contact force that causes the airway wall to deform or a combination of both in order to provide a continuous circumferential sealing band to prevent air leakage in to a targeted portion of the lung under pressure differentials normally experienced in the lung. When implanted in a target airway, a structural frame may be adapted to impart an outward contact force that may expand the airway wall no more than 20% which is expected to provide strong contact and a good air seal while avoiding trauma to the tissue that otherwise could cause excess formation of granulation tissue. The wall contact region may have circumferentially extending struts 308 that press the membrane 312 against the airway wall in a way that disrupts or prevents longitudinal membrane folds from creating paths through which air may leak. For example, the interconnected struts 308 may apply contact pressure to the airway wall continuously around the circumference of the airway (including zig-zagging, diagonal, helical, or rows of diamonds patterns). Interconnected struts may interact with a sealing membrane to apply contact pressure and contact surface area between the wall contact region and airway wall. In some situations a sealing membrane may have a wrinkle or fold when the lobar valve is implanted in an airway, for example due to an irregular shape or size of the airway. The circumferentially continuous arrangement of the interconnected struts may disrupt any wrinkles or folds in the membrane to facilitate an air seal.

Optionally, a wall contact area 309 in its unconstrained state may be barrel shaped (e.g., have a wider middle than proximal and distal ends) or be flared (e.g., have a larger diameter distally than proximally), which may facilitate creating a good contact region and seal with the airway wall.

The wall contact region 309 of the structural frame 302 provides a scaffold for the membrane 312, which is affixed to the interconnected struts 308 of the frame, for example by dip coating, adhesive, or other form of bonding. The structural frame may be collapsed to its contracted delivery state in an orderly fashion that does not damage the membrane.

Spokes

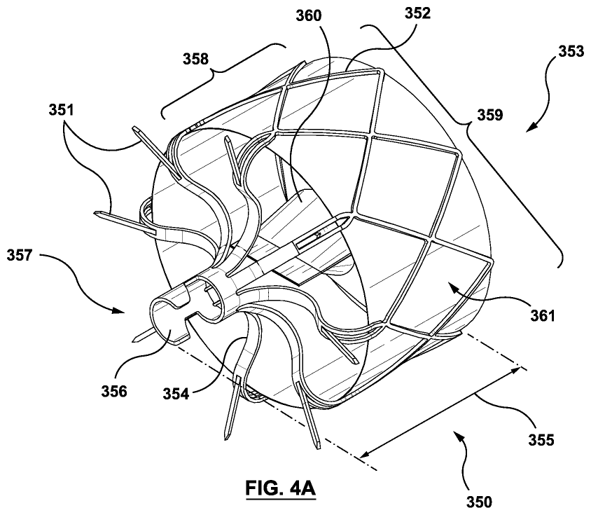

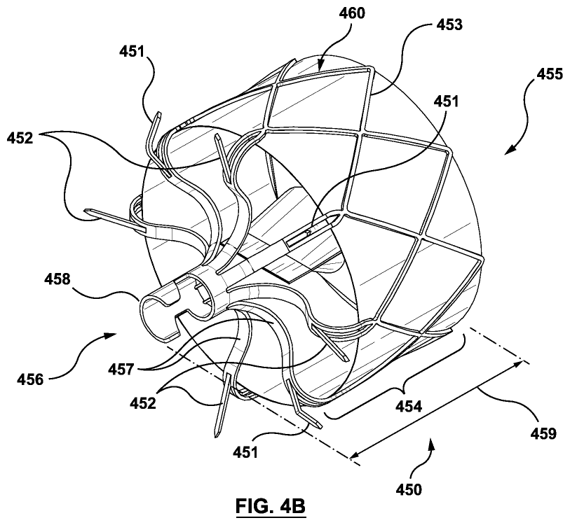

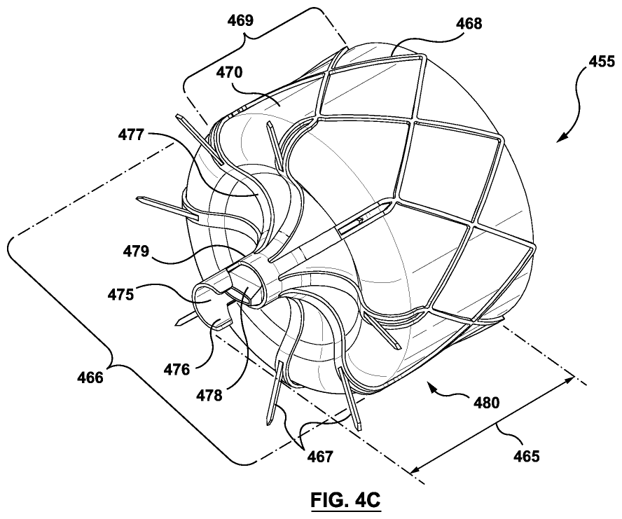

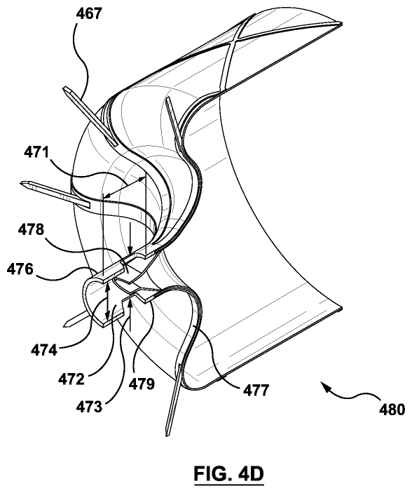

Still referring to FIG. 3A as an example, a wall contact region 309 may be connected to a coupler 304 at the proximal end 305 of the device 300 by spokes 307. As shown in an expanded state the spokes 307 may radially expand from the coupler 304 having a narrow diameter to the wall contact region 309 having a larger diameter. In its compressed delivery the spokes 307 may transfer force (e.g., axially directed push or pull translation or rotation) applied to the coupler 304, for example by a delivery tool attached to the coupler, to the wall contact region 309. The spokes may impart an elastic force radially outward to the wall contact region but shall not apply sufficient force to interrupt the air sealing function of the wall contact region. When a device 300 is in its expanded state and a delivery sheath is advanced over the coupler 304 the force applied by the delivery sheath to the spokes 307 may cause the spokes to radially contract and collapse the wall contact region 309 allowing the device to be fully pulled back into the delivery sheath or to at least partially reduce the diameter of the wall contact region. This may be used to remove contact force with the airway wall to facilitate repositioning of the device. Optionally, spokes 307 may have a proximal take-off section 314 that is shape set with a concave curve or lesser angle to the coupler than the rest of the spokes which may facilitate collapse of the device by advancing a delivery sheath which will first apply force to the take-off section to begin collapsing the spokes. In some embodiments, as shown in FIG. 4A, 4B, 4C and 5A spokes 354, 457 501 may have an “S” shaped curve that positions the coupler 356, 458, 502 longitudinally closer to the wall contact region 358, 454, 503 thereby reducing to overall length 355, 459, 504 of the device when it is in its expanded state.

Valve Housing

Referring to FIG. 4C as an example, an implantable endobronchial valve 480, such as a lobar valve, optionally may have a valve housing 479 that functions to shroud and frame a one-way valve 478 and is made from a structural frame. The valve housing 479 may be positioned on the structural frame between the spokes 477 and the coupler 476.

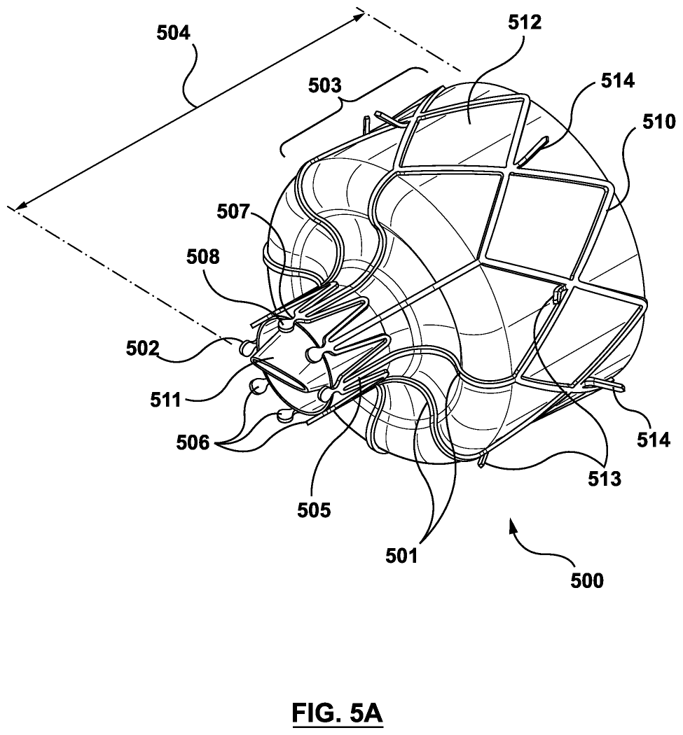

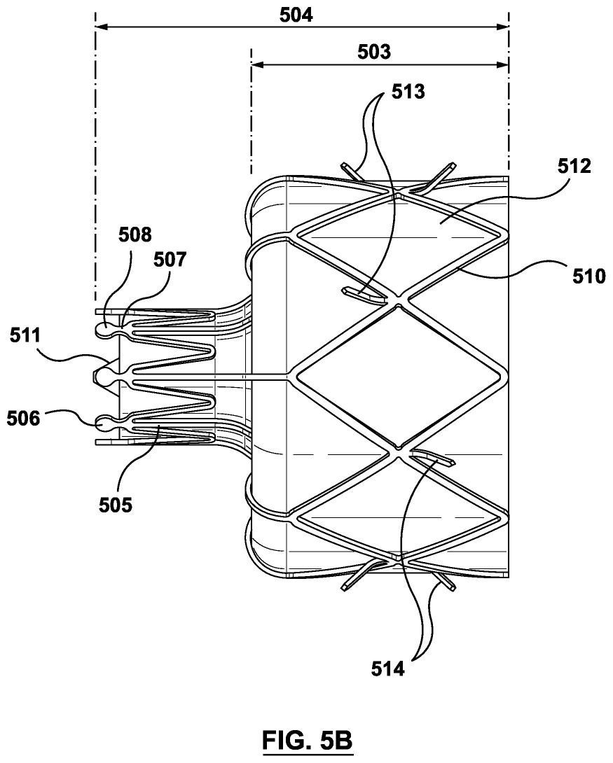

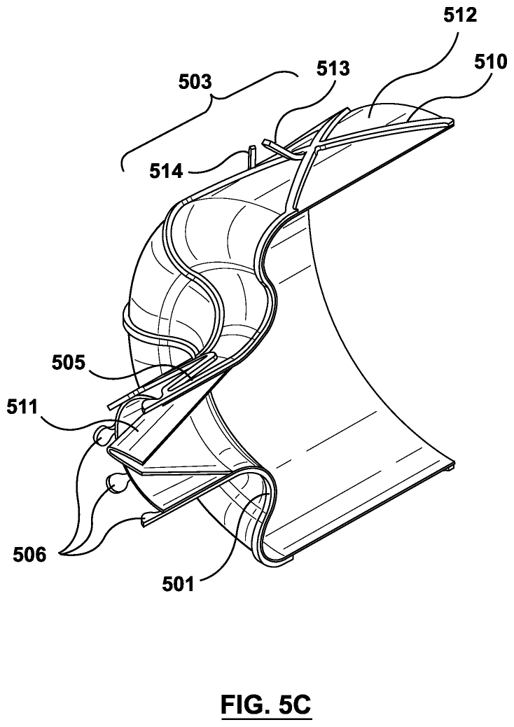

In another embodiment as shown in FIG. 5A, 5B and 5C a device 500 may have a valve housing 505 that is radially transformable from a contracted delivery state to an expanded state. As shown in FIGS. 5A, 5B and 5C the expandable valve housing 505 is in its expanded state. FIG. 7A shows the device of FIG. 5A connected to a delivery shaft 540 wherein the coupler 502 of the device is mated with a coupler 542 of the delivery shaft. A delivery sheath 541 constrains the expandable coupler 502 in its contracted delivery state wherein the expandable valve housing has a constrained diameter 544. FIG. 7D shows the device 500 released from the driveshaft and delivery sheath where the expandable coupler 502 and valve housing 505 is allowed to elastically transform to its unconstrained expanded state wherein its diameter 543 (e.g., a diameter in a range of 3 mm to 4 mm) is larger than the constrained diameter 544 (e.g., a diameter in a range of 2 mm to 2.8 mm shown in FIG. 7A).

Coupler

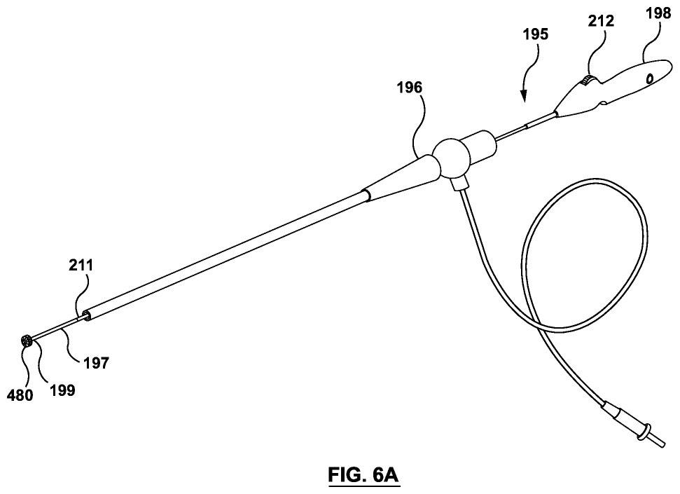

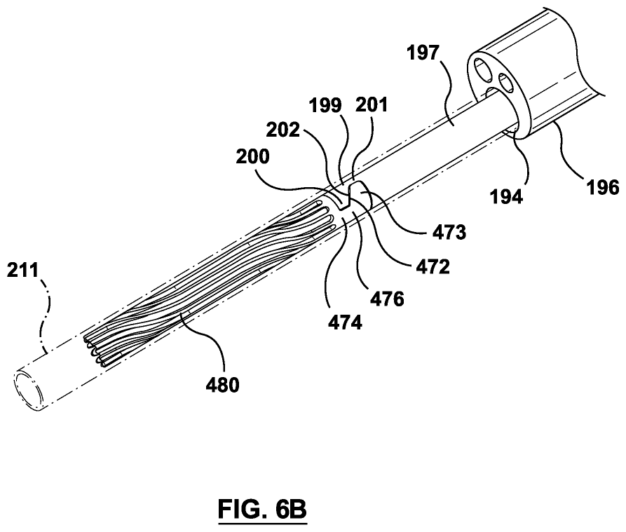

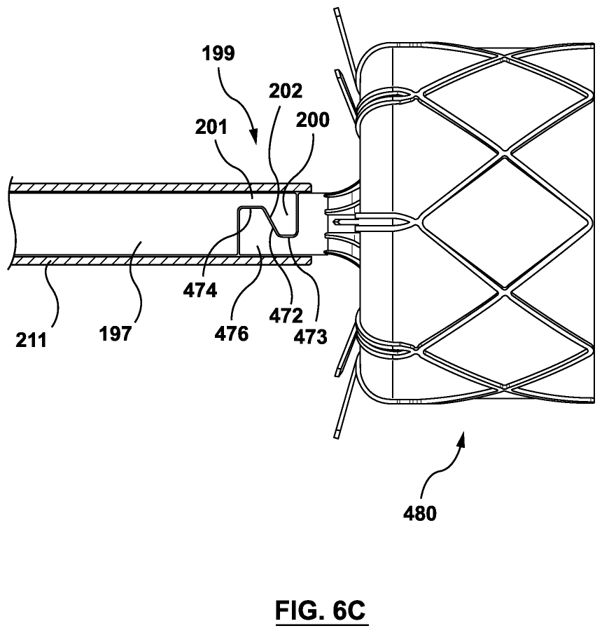

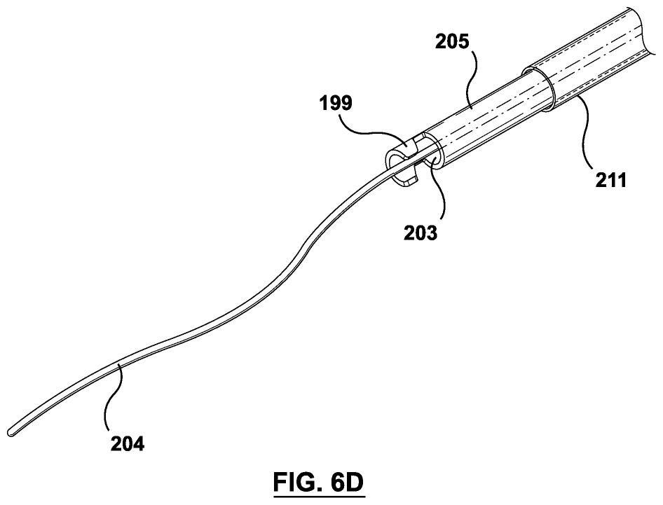

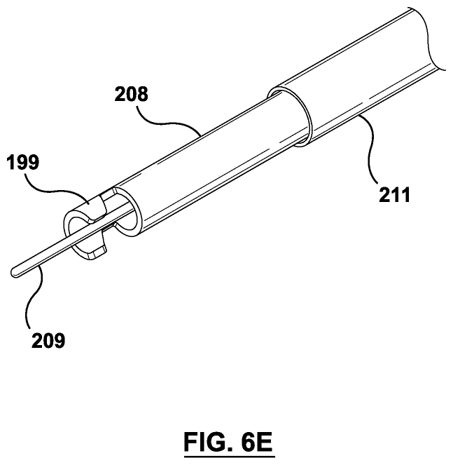

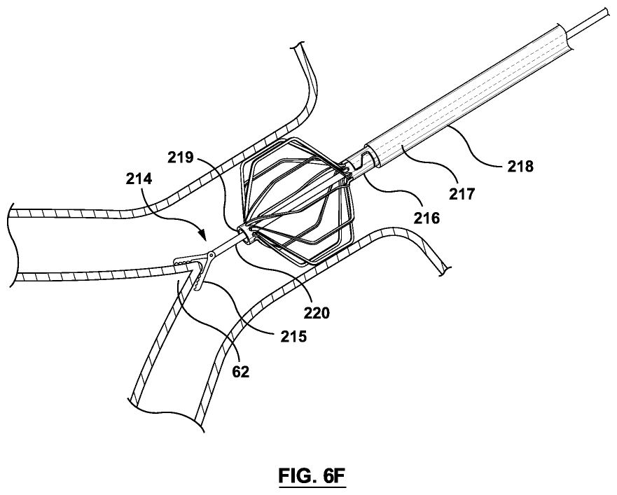

A lobar valve may further have a coupler positioned at a proximal end of the device that functions to mate with a coupler of a delivery shaft and release from the coupler of the delivery shaft upon actuation by a user. The coupler may be part of a structural frame and made by laser cutting a tube. For example, the lobar valve's coupler may be held in a mated configuration with a coupler of a delivery shaft when contained within a delivery sheath and when the sheath is retracted the couplers disengage. An actuator (e.g., rotary dial, trigger, slider, button) controllable by a user for example on a handle connected to the delivery sheath and delivery shaft may control the relative position of the delivery shaft and sheath to control release of the couplers. A device coupler may remain attached to a delivery shaft coupler when radially constrained by delivery sheath by maintaining longitudinal alignment of the couplers (e.g., FIG. 6C) or by maintaining an expandable coupler of a lobar valve in a contracted delivery state that mates with a delivery shaft coupler (e.g., FIG. 7A). When attached the coupler transmits motion of the delivery shaft to the implantable valve including longitudinal translation distally, proximally and rotation around longitudinal axis.

In some embodiments having a valve housing the coupler may be connected to the valve housing. For example, as shown in FIG. 4C the coupler 476 is directly proximal to the valve housing 479. The coupler in this embodiment has a notch 475 cut from the tube. The notch has a proximal neck 474 that is narrower than a distal flare 473 (see FIG. 4D). A transition 472 from the neck 474 to distal flare 473 may be sloped, for example angled to facilitate release or reconnecting of the coupler 476. The coupler 476 may have a length 471 in a range of about 0.11″ to 0.12″ (2.8 to 3 mm). The distal end of the coupler may be a tubular section that is uncut around its circumference and that connects to the structural frame's spokes 477. The tubular section may function as a valve housing 477.

In another example, as shown in FIG. 5A and 7A to 7D the coupler 502 is directly proximal to an expandable valve housing 505. The coupler 502 has multiple (e.g., 2 to 10, 8) coupling members 506 that fit in negative space of a delivery shaft coupler 542 and are held in a mating position by a constraining sheath 541. The coupling members 506 may be rounded at their terminus, which may help to avoid a risk of injuring the airway. Each coupling member 506 may have a neck 507 distal to a head 508 wherein the neck is narrower than the head and both are sized to fit in a negative space of a neck 545 and head 546 of a delivery shaft coupler 542 (see FIG. 7D). This allows the couplers 542 and 502 to remain locked and for movement to be transferred from the delivery shaft to the lobar valve when the couplers are constrained in a delivery sheath yet the couplers may disengage when the delivery sheath is retracted.

Covering/Seal

Lobar valves disclosed herein may further have at least one membrane (470 in FIG. 4C) connected to the structural frame that functions to create an air seal of the lobar bronchus permitting air to flow only or at least predominantly through a one-way valve 478. The material of the sealing membrane may further function to resist tissue ingrowth so the lobar valve may be safely removed after a prolonged period of remaining implanted. The material may be made from a material or have a layer that avoids it from sticking to itself, which facilitates transformation of the lobar valve from a collapsed delivery state to an expanded deployed state.

The membrane connected to the structural frame may be made from a thin, flexible, durable, foldable, optionally elastic material such as urethane, polyurethane, ePTFE, silicone, Parylene or a blend of multiple materials. The membrane may be made by insert molding, dip coating or spray coating a mold or other manufacturing methods know in the art of medical balloon or membrane manufacture. It may be bonded to the frame for example by coating the frame, laminating over the frame, dip coating, spray coating, heat staking, bonding with adhesive, or sewing. Referring to FIG. 4C as an example a membrane 470 may cover the wall contact region 469 of the structural frame 468 and at least a portion of a luminal covering region 466 to disallow air from flow through a lumen of the bronchi except for through the one-way valve 478 and impede air from leaking around the edges between the wall contact region and an airway wall. As shown in FIG. 4C a luminal covering region 466 may be on a proximal side of a wall contact region 469 and the membrane 468 may optionally be bonded to spokes 477. Alternatively, as shown in FIG. 4A a luminal covering region 359 may be on a distal side 353 of a wall contact region 358. This configuration also permits the device to seal airflow when positioned in an irregular shaped bronchus or when positioned offset from the axis of the bronchus.

The sealing membrane may be positioned inside the cavity formed by the structural frame and bonded to the inner surface of the structural frame, such as shown in FIG. 5A. Alternatively the sealing membrane may be positioned and bonded outside the structural frame. Alternatively, a sealing membrane may have an inner membrane layer bonded to the inner surface of the structural frame as well as an outer membrane layer bonded to an outer surface of the structural frame wherein the inner and outer layers may be bonded to one another between interconnected struts or spokes thus encapsulating at least a portion of the structural frame.

Airflow 181 as shown in FIG. 7D flows from the lobe distal to the device 500, through a valve 511, and out of the lung. The sealing membrane 512 in combination with the one-way valve 511 impedes air from flowing the opposite direction into the lobe. Optionally, the membrane may also form the one-way valve, or alternatively a one-way valve may be a separate structure connected to a structural frame or sealing membrane.

Portions of the sealing membrane 512 framed by interconnected struts 510 of a wall contact region 503 may be flexible and have slack that functions to facilitate air sealing by billowing out and applying contact pressure with the airway wall over a surface area defined by the sealing membrane portions when air is passing through the device or a pressure difference is higher within the device.

The sealing membrane and structural frame, in particular the wall contact region, form a contact surface area that is continuous around a circumference of a targeted airway wall.

In an alternative embodiment of a seal the seal may have channels that intentionally allow air to pass the seal in either direction initially after the device is implanted and gradually close to block air passage except for through a valve. For example, the channels may be positioned on the seal surface next to the airway wall and over time (e.g., a few weeks) become plugged with mucus that naturally exists in the airway. Gradual or delayed sealing could delay the evacuation of trapped air and subsequent lobar volume reduction so that shifting of the lobes of the treated lung occurs more gradually, which may be less likely to have adverse events such as pneumothorax or injury to healthy lung tissue.

Optionally, a membrane may deliver a chemical agent released slowly over time. For example, the membrane may deliver an antiseptic, antimicrobial or other agent, which may reduce the risk of infection, pneumonia, rejection or other complication. For example, a membrane may be impregnated with an agent such as an antifungal, antibacterial, antimitotic, or anti-inflammatory agent that may improve patient response to implanting the device.

Valve

The device is adapted to provide a seal that does not allow air to flow, or at least substantially increases resistance to airflow through the targeted airway except for through a one-way valve. The sealing function is achieved by a membrane connected to the structural frame and the sealing membrane may also form the one-way valve. Alternatively, a valve may be a separate structure bonded to the sealing membrane or structural frame. Generally, a valve is adapted to allow air to flow at least predominantly in one direction, from the affected lobe and not into it. In other words, as illustrated in FIG. 7D a lobar valve 500 restricts airflow 181 to flow in a direction from distal side of the lobar valve to the proximal side.

Optionally, a valve material may be impregnated with an agent such as an antifungal, antibacterial, antimitotic, or anti-inflammatory agent that may improve patient response to implanting the device.

As an example, referring to FIG. 4D which is a cut away view showing the device of FIG. 4A, a one-way valve 478 may be made from a flexible, non-stick material such as an elastomeric material, urethane, polyurethane, ePTFE, silicone, Parylene or a blend of multiple materials. The one-way valve 478 may be a duckbill valve having a somewhat funnel shape that transitions from a distal flared end to a proximal closing end. The distal flared end may be tubular having an outer diameter that connects with the luminal covering region 466 of the sealing membrane 470 and in some embodiments fits within a valve housing 479. The distal flared end may have a diameter in a range of 1 mm to 4 mm (e.g., 3 mm to 4 mm). The duckbill valve 478 includes a pair of opposed, inclined walls having ends that meet at lips at the proximal closed end. The lips meet at two opposed corners and may be pinched flat. The walls can move with respect to one another so as to separate at the lips and form an opening through which fluid can travel. When exposed to fluid flow in a direction represented by the arrow 181 in FIG. 7D at a cracking pressure, the walls separate from one another to form the opening through which the fluid may flow. When exposed to fluid flow in an opposite direction the lips remain closed and prevent fluid from flowing through the duckbill valve. Alternatively, other forms of one-way valves known in the art of medical devices may be used. Optionally, the lips may be normally opened at least a small amount when there is no pressure differential across the valve, which may reduce or eliminate the cracking pressure and reduce an opening response time.

Retention Mechanism

A lobar valve may have a retention mechanism such as barbs, radial compression, or radial interference. The retention mechanism functions to keep the device situated and oriented in the targeted position of the patient's airway. The device may be removed by applying force (e.g., pulling, torqueing) to the coupling element to overcome the retention force. Alternatively, the retention mechanism may be released from the airway by collapsing the lobar valve.

FIG. 2 shows a structural frame 242 having interconnected struts 249 forming a zig-zag pattern around a wall contact region 248. At the distal end 243 of the device the interconnected struts may terminate in a connection 250 that functions to apply outward contact force to a targeted airway wall and also a force vector aimed toward the distal direction 243. For example the terminal connections 250 may be flared outward. Optionally, the terminal connections 250 may have a rounded end 251 that increases surface area and reduces a risk of puncturing the airway wall or injuring it. The terminal connections 250 and/or the rounded ends 251 may act as a retention mechanism by applying friction and force directed radially outward.

The lobar valve 241 has a coupler 245 which may be formed in a cylindrical section 244 which has an open section to form a structure which connects with a matching coupler of a delivery shaft. The cylindrical section includes an annular collar 246 that joins the coupler 245 to the spokes 247 and provides structural supports for the spokes.

Other embodiments of lobar valves 300, 260, 300, 350, 450 are shown in FIG. 3A to 5C. The lobar valves have radially outward facing barbs 301, 261, 351, 451, 452, 467, 513, 514. Referring to FIG. 4C as an example, the lobar valve 480 comprises a structural frame 468 that has radially extending barbs 467 that are deployed when the structural frame is expanded to its deployed state. The barbs 467 are part of the structural frame and are laser cut from the tube and shape set in its expanded state. In one embodiment the barbs have tips that are not sharp (e.g., square cut, rounded) and they function as a retention mechanism by wedging into groove or an uneven surface of the airway wall, for example grooves made by cartilage rings or plates, or simply by concentrating frictional force against the airway wall. In addition to radially extending the barbs may aim in direction angled proximally (e.g., as in FIG. 4A and 4C) or distally (e.g., as in FIG. 3A and 3B) or a device may have barbs that face both proximally and distally (e.g., as in FIGS. 4B and 5A), which may produce vector forces against the airway wall that further resist longitudinal migration through an airway. FIG. 4B shows an embodiment of a lobar valve 450 having radially extending barbs 451 angled toward a distal end alternating with radially extending barbs 452 angled toward a proximal end that are integrated into a structural frame 453.

Barbs 451, 452 may be positioned on a wall contact region 454 of a lobar valve 450 optionally, at the proximal end, distal end or somewhere in between but preferably at the distal end since this end contacts the airway wall first when deployed from a delivery state.

In a constrained delivery state the barbs 451, 452 may be retracted and flush with the spokes 457 and interconnected struts 460, allowing the device to be advanced from a delivery sheath.

The barbs 451, 452 may protrude from the wall contact region 454 in a range of 0.25 mm to 1 mm.

Regardless of the retention mechanism embodied, a lobar valve 450 may be implanted and before removing the delivery tool and bronchoscope, a pull force test may be applied to the device to ensure it has been sufficiently anchored in place. With the delivery tool connected to a grasping mechanism of an implanted lobar valve, the pull force may be conducted by applying a gentle pull force on the delivery tool. A force gauge may indicate the amount of force applied to the lobar valve. If the valve becomes dislodged below a predetermined force, the retention mechanism of the stent may not suit the current implantation, a different sized device may be required, or the device may need to be repositioned.

Example Embodiments

FIG. 3A shows an example of a lobar valve 300 having a structural frame 302 with a wall contact region 309 having interconnected struts 308 forming a diamond cell pattern. The structural frame 302 has a coupler 304 at its proximal end 305 connected to a proximal collar 306. Strut spokes 307 connect to and extend radially from the proximal collar 306. In a wall contact region 309 intended to contact the airway wall the strut spokes each divide into branches 308 that converge at a distal end connection point 310 and are connected to adjacent branches at cell connection points 311. This structure forms diamond-shaped cells 312 that are connected to one another at the cell connection points 311, to the strut spokes 307 at the cells' proximal end, and are free floating at the distal connection points 310. The structural frame 302 has barbs 301 angled radially outward and distally that deploy when the frame is expanded. A sealing membrane 316 is bonded to the structural frame around the wall contact region 309 and extends inward to block a luminal covering region 315 at the distal end of the device where it is connected to a valve 313 that is held within the expanded structural frame's wall contact region 309. This design may function to provide support for the sealing membrane 316 and apply an air seal between the device and airway wall as well as allow the sealing membrane 316 to billow between interconnected struts 308 during inhalation (e.g., wherein air pressure is higher on the proximal side 305 than the distal side 303 of the device) to provide an additional air sealing function. The diamond shape of the cells 312 and cell connection points 311 may also improve the air seal between the device and airway wall by interrupting any folds that may form in the membrane seal.

FIG. 3B shows a lobar valve 260 that is similar in design to the device 300 of FIG. 3A however the sealing membrane 276 on the distal side 263 of the lobar valve 260 covers both a proximal covering region 275 where the membrane covers the spokes 267 and a distal cylindrical region 269 where the membrane covers struts 268. The sealing membrane forms an air barrier because it includes both the proximal covering region 275 which substantially blocks the airway and the distal cylindrical region 269 that forms an expandable wall contact region 269 to seal against the natural walls of the airway. The membrane 276 may continue proximally to form a one-way valve 273 which may be positioned and supported by a valve housing 266. The cylindrical valve housing 266 may also form a coupler 264.

In lobar valve 260, the one-way valve 273 and the membrane 276 may be an integral component, e.g., a single piece component, such as formed of a layer of plastic.

In the lobar valve 260, the spokes 267 may include barbs 261 extending radially outward of the spokes and the expandable wall contact region 269. The structural frame is a mesh formed by proximal struts 268 and distal struts 262 connected at junctions 271. The distal ends 270 of the struts 262 may be rounded or curved, and support the distal circumferential edge of the sealing membrane.

FIG. 4A shows another example of a lobar valve 350 having a structural frame 352 that is open at the distal end 353. This lobar valve 350 is similar to the lobar valve 300 shown in FIG. 3A however the frame 352 has spokes 354 that radially extend when the device is in its expanded state and have an “S” shaped curvature as shown that reduces the overall length 355 by positioning the coupler 356 closer to the center of the device as compared to the design of FIG. 3A. A reduced overall length may allow a better fit in a short lobar bronchus and may be easier to cough out if the device becomes dislodged. The curved strut spokes 354 may provide a greater contact force with the airway wall compared to the design of FIG. 3A. Optionally the structural frame has barbs 351 formed from the strut spokes 354 (e.g., laser cut). As shown the barbs 351 are angled outward and proximally. Alternatively as shown in FIG. 4B barbs 452 and 451 radially extend beyond the wall contact region 454 and alternate in the direction they are angled toward. Barbs 451 are shown angled toward a distal end 455 while barbs 452 are shown angled toward the proximal end 456. Alternating barbs may function to anchor the device 450 in a lobar bronchus and resist movement in and out of the airway and may further retain orientation.

The sealing membrane for lobar valve 350 may include a cylindrical section 361 which is attached in inside surfaces of the struts 352 and a one-way valve 360. The sealing member thus forms a barrier layer extending from the struts 352 radially inward to the one way valve.

FIG. 4C shows another example of a lobar valve 480 that is similar to the device shown in FIG. 4A however the valve 478 is positioned at the proximal end and is supported in a valve housing 479. The membrane 470 is bonded to the interconnected struts in the wall contact region 469 and the spokes 477 in the luminal covering region 466 and forms the one-way valve 478. FIG. 4D is a longitudinal cross section of the device 480 of FIG. 4C.

FIG. 5A shows another embodiment of a lobar valve 500 wherein a valve housing 505 is expandable. The valve housing is made from interconnected struts that are transformable from a contracted delivery state to an expanded deployed state when unconstrained by a delivery sheath. A one-way valve 511 supported by the valve housing is made from a flexible membrane (e.g., an elastomer) that can collapse (e.g., by folding) to fit in the valve housing's delivery state and expand along with the valve housing to a deployed state. A coupler 502 also expands. As shown the spokes 501 have an “S” shaped contour but alternatively may be straighter as in the spokes 307 shown in FIG. 3A. Barbs 513, 514 are shown alternating in direction and positioned in the middle of the wall contact region 503 but alternatively may be arranged as other barbs disclosed herein.

Delivery Tool

As shown in FIGS. 6A and 6B a delivery tool 195 for delivering a lobar valve (e.g., 480) through a working channel of a bronchoscope 196 may have a delivery shaft 197, which may be a flexible, elongate, tubular or rod structure, with a coupling element 199 at its distal end that is shaped to couple with the coupler (e.g., 476) of the lobar valve, a delivery sheath 211, and a handle 198 at its proximal region. For example, the coupling element 199 may have a cut or groove or a negative space that mates with a positive space of the lobar valve's coupler 476. For example, to mate with a coupler 476 having a notch neck 474, a notch flare 473 and transition slope 472, the coupling element 199 may have a flare 200, a neck 201 and a transition slope 202 transitioning between the neck 201 and flare 200. A delivery shaft may be flexible to bend and navigate through a bent bronchoscope in a tortuous airway yet be longitudinally and circumferentially non-compliant to resist stretching, compression or kinking so it transmits motion from the proximal end (e.g., handle) to the coupling element 199 and to the lobar valve. A delivery shaft may be made from a polymer and have an embedded laser cut tube or tight wire coil.

An alternative embodiment of a delivery shaft 205 as shown in FIG. 6D, may have a central lumen 203, which may be used for delivery over a guidewire 204 or to pass over or deliver other instruments such as an endoscope. Optionally as shown in FIG. 6E a delivery shaft 208 may have a mandrel 209 extending distally, which may be used to hold a valve (e.g., 87) to the delivery shaft 208, to add coupling force, to target a coupler 264 of a lobar valve when retrieving it or to adjust its position.

Optionally, the delivery tool may have a delivery sheath 211 used in conjunction with the delivery shaft 197, 205, 208. As shown in FIG. 6B the sheath may constrain the mating coupler 476 and coupling element 199 into a locked position when they are housed in the sheath. Retracting the sheath or advancing the mating coupler 476 and coupling element 199 out of the sheath may allow movement of the mating elements to release. The sloped transition 202 and 472 may facilitate release or reconnecting of the mating elements. The sheath may constrain the valve in a delivery state during delivery through a working channel as shown in FIG. 6B. A distal section (e.g., about 10 cm of the distal end) of the delivery sheath may be relatively more flexible allowing it to bend and traverse a bronchoscope that is bent at its distal end to navigate a tortuous airway. The delivery sheath may be non-compliant over its full length to resist compression or stretching. The delivery sheath may be circumferentially non-compliant at least at its distal end so it can contain and constrain a lobar valve in its contracted delivery state. A laser cut steel tube may be embedded in a polymer such as Pebax at its distal section to provide hoop strength and circumferential non-compliance. The delivery sheath may be made of a polymer such as Pebax or polyimide with an embedded wire braid or wire coil to resist compression, stretching or kinking. The delivery sheath 211 may have an outer diameter sized to slidably pass through the bronchoscope working channel 194 (e.g., to fit a 2.8 mm the sheath may have an outer diameter in a range of 2.0 mm to 2.7 mm). The sheath may have an inner diameter in a range of 1.5 mm to 2.5 mm.

Optionally, a delivery tool may have a forceps tool 214 that slidably passes through a lumen 217 of a delivery shaft 216 and the forceps tool may pass through an opening in a one-way valve of a lobar valve as shown in FIG. 6F to grab on to tissue such as an airway carina 62. The forceps tool 214 may have forceps 215 at a distal end of the forceps tool that are actuated by an actuator on a handle on a proximal section of the forceps tool (not shown). In use during an implant procedure, the forceps 215 may be advanced through the delivery shaft lumen 217 and valve, for example with the valve contained in its delivery state within a delivery sheath 218, and the forceps may be actuated to grasp tissue such as an airway carina immediately distal to a lobar bronchus. A flexible shaft 220 connected to the forceps 215 may be used as a rail to assist in delivery of the valve. The membrane and valve are omitted from FIG. 6F to show the structural frame however, it is to be understood that a lobar valve will have a sealing membrane and valve as shown as several embodiments in other figures herein. The delivery shaft 216 may be advanced relative to the delivery sheath 218 to push the valve out of the sheath deploying it to its expanded state. This motion may be accomplished by retracting the sheath with respect to the forceps tool and lung tissue, or by advancing the delivery shaft while holding the sheath still relative to the forceps or a combination of these. Optionally, the delivery shaft may be lockable to the forceps tool at the proximal end of the delivery tool, for example with a collet or set screw, which may secure translational position of the valve with respect to the forceps tool and thus with respect to the lobar bronchus when the forceps is grasping lung tissue. Then the sheath may be retracted, optionally with an actuator 212 on the delivery tool (see FIG. 6A).

In an alternative embodiment of a delivery tool, a coupler 542 shown in FIG. 7A is configured to mate with an expanding lobar valve coupler (e.g., 502 of device 500).

Optionally, the delivery tool may have a handle 198 at a proximal region that has an actuator (e.g., thumb lever) that controls a sliding translational movement of the shaft 197 with respect to the sheath 211 facilitating one-handed control for advancing a valve out of a sheath or retracting it into the sheath. For example, a sheath 211 may be connected to the handle body and a shaft 197 may be slidably engaged in the sheath and connected to a gear that is movable (e.g., rotation or translation) within the handle and moved by a mating gear connected to an actuator such as a thumb lever, slider, or rotary dial. The handle may have one or more actuators that move the delivery shaft and control the position of the lobar valve from a fully contained position as shown in FIG. 7B to a partially deployed position with the couplers connected as shown in FIG. 7C, to a fully deployed and released position as shown in FIG. 7D. For example, a delivery tool 195 and lobar valve 500 may be provided in a sterilized package with the delivery shaft 540 positioned in a first position (stage 1) with the couplers 542, 502 locked together and the device 500 partially deployed as shown in FIG. 7A. A first actuator may be used to pull the device 500 into the delivery sheath (FIG. 7B). The first actuator may be used to advance and retract the device to partially deploy it. This step may be used to assess position and fit within a target airway while visualizing deployment through a lens 193 of the bronchoscope 196. The first actuator may stop at the position of stage 1 before fully releasing the device. A second actuator such as a trigger may be used to fully retract the delivery sheath or to unlock the first actuator from stage 1 and allow it to advance further and release the device 500 (FIG. 7D). The first and second actuators may be ergonomically oriented on the handle 198 to be used with one hand for example the first actuator may be oriented for use with a thumb and the second actuator may be oriented for use with an index finger of the same hand. Optionally, the delivery sheath and delivery shaft may be connected to a rotational actuator on the handle that rotates them while maintaining a handle position that is comfortable for the operator's hand.

Kit

Optionally the valve may be provided preloaded in a delivery sheath, optionally disposable, in its constrained delivery state and coupled with a delivery shaft as shown in FIG. 6B. Alternatively, a lobar valve may be provided coupled to a delivery tool with a delivery sheath holding the couplers together but with the rest of the lobar valve advanced out of a delivery sheath in its unconstrained state for example as shown in FIG. 7A. The assembly may be provided contained in a sterilized package with instructions for use. A lobar valve provided partial deployed may facilitate visual inspection and avoid material deformation caused by prolonged constraint.

Delivery

A method of use may involve the following delivery steps:
From a CT scan measurements confirm intended valve placement location, target airway diameter and length;
Visually inspect the lobar valve provided coupled to a delivery system (FIG. 7A);
Advance a bronchoscope through the patient's endotracheal tube to the targeted lobar airway;
Retract the lobar valve into the delivery sheath and advance the pre-loaded lobar valve delivery system distally through a working channel of the bronchoscope;
Advance the distal end of the delivery system distally out of the working channel to a desired valve position in the target airway (FIG. 7B);
While holding the bronchoscope in position relative to the airway retract the delivery sheath proximally relative to the lobar valve to the expanded but coupled position (stage 1, FIG. 7C);
Visually inspect position, fit, alignment, and seal through the lens of the bronchoscope. Pull gently on delivery system to confirm mechanical anchoring or engagement of valve against airway wall;
Confirm respiratory motion of airway has stopped indicating the lobar valve is occluding the airway;
If position, fit, alignment, seal and anchoring are not satisfactory push or pull the delivery system to adjust;
If position, fit, alignment, seal and anchoring are still not satisfactory retract the lobar valve back into the delivery sheath;
Reposition the delivery sheath and lobar valve;
If the position, fit, alignment, seal and anchoring are satisfactory retract the delivery sheath to stage 2 position which fully unsheathes the lobar valve and disengages the coupler of the delivery system from the valve's coupler;
Remove the delivery system;
Visually inspect the lobar valve through the lens of the bronchoscope;
Remove the bronchoscope.

While at least one exemplary embodiment of the present invention(s) is disclosed herein, it should be understood that modifications, substitutions and alternatives may be apparent to one of ordinary skill in the art and can be made without departing from the scope of this disclosure. This disclosure is intended to cover any adaptations or variations of the exemplary embodiment(s). In addition, in this disclosure, the terms “comprise” or “comprising” do not exclude other elements or steps, the terms “a” or “one” do not exclude a plural number, and the term “or” means either or both. Furthermore, characteristics or steps which have been described may also be used in combination with other characteristics or steps and in any order unless the disclosure or context suggests otherwise. This disclosure hereby incorporates by reference the complete disclosure of any patent or application from which it claims benefit or priority.

1. A flow control device for a bronchial passageway comprising:
a one-way valve;
a hollow structural frame housing the one-way valve, wherein the structural frame is expandable from a collapsed configuration to an expanded configuration; and
a sealing membrane mounted to at least a distal portion of the structural frame, wherein the sealing membrane forms a wall spanning the distal portion and configured to occlude at least a portion of an airflow passage through the flow control device, and the one-way valve has an inlet at the wall and an outlet within the hollow structural frame.

2. The flow control device of claim 1 further comprising barbs extending radially outward from the structural frame while in the expanded configuration.

3. The flow control device of claim 2 wherein the barbs extend at an angle acute to a longitudinal axis of the flow control device.

4. The flow control device of claim 2, wherein some of the barbs are angled towards a distal end of the flow control device and others of the barbs are angled towards a proximal end of the flow control device.

5. The flow control device of of claim 2, wherein at least some of the barbs extend from spokes of the hollow structural frame.

6. The flow control device of claim 2, wherein at least some of the barbs extend from a middle section of the hollow structural frame.

7. The flow control device of claim 2, wherein at least some of the barbs extend from a cylindrical section of the hollow structural frame, wherein the cylindrical section is at a distal portion of the flow control device.

8. The flow control device of claim 1, wherein a width of the hollow structural frame in the expanded configuration is in a range of 7 mm to 12 mm.

9. The flow control device claim 1, wherein a length of the flow control device in the expanded configuration is in a range of 5 mm to 15 mm.

10. The flow control device of claim 1, wherein the hollow structural frame while in the expanded configuration, includes an expanded cylindrical section at a distal section of the flow control device and the outlet of the one-way valve is in the expanded cylindrical section.

11. The flow control device of claim 10, wherein the sealing member is confined to the expanded cylindrical section.

12. The flow control device of claim 10, wherein the sealing member covers the expanded cylindrical section and the flow control device further includes spokes included in the structural frame.

13. The flow control device of claim 1, wherein the hollow structural frame in the collapsed configuration has a diameter no greater than 2.6 mm.

14. The flow control device of claim 1, wherein the hollow structural frame in the collapsed configuration has a diameter in a range of 2 mm to 2.6 mm.

15. The flow control device of claim 1, wherein a ratio of a length to a width of the hollow structural frame in the expanded configuration is in a range of 0.28:1 to 0.54:1.

16. The flow control device of claim 1, wherein a ratio of a width of the hollow structural frame in the expanded configuration to the width in the collapsed configuration is in a range of 4:1 to 7:1.

17. The flow control device of claim 1, wherein the flow control device includes a coupler at a proximal end of the device.

18. The flow control device of claim 1, wherein the flow control device includes a coupler at a proximal end of the device, and the coupler is configured to connected to a corresponding coupler of a shaft of a delivery device.

19. The flow control device of claim 18 wherein the coupler is formed in a laser cut tube forming a proximal portion of the flow control device.

20. The flow control device of claim 19 wherein the laser cut tube has a wall thickness in a range of 0.11 mm to 0.17 mm.

21. The flow control device of claim 19 wherein the coupler comprises multiple coupling heads each connected with necks to coupling members and wherein the coupler is transformable from a contracted delivery state to an expanded state.

22. The flow control device of claim 21 wherein the coupling members form an expandable valve housing surrounding a one-way valve.

23. An assembly of an air flow control device and an insertion tool for a bronchial passageway comprising:
an air flow control device, wherein each of the air flow control devices includes:
a one-way valve;
a hollow structural frame housing the one-way valve, wherein the structural frame is expandable from a collapsed configuration to an expanded configuration;
a sealing membrane mounted to at least a distal portion of the structural frame, wherein the sealing membrane forms an enclosed wall defining at least a portion of an airflow passage through the flow control device, and the one-way valve is included in the airflow passage, and
a first coupler at a proximal end of the airflow control device;
a delivery sheath configured to be positioned in a bronchial passageway, wherein the delivery sheath includes a distal end, wherein the air flow control device, while in the collapsed configuration, is within the delivery sheath;
a delivery shaft within the delivery sheath and extends through the delivery sheath towards the distal end; and
a second coupler at the distal end of the delivery shaft, wherein the second coupler is configured to securely engage the first coupler while the air flow control device is at least in part in the delivery sheath,
wherein the delivery shaft is configured to advance through the delivery sheath to push the air flow control device from the distal end of the delivery sheath and into the bronchial passageway, and
wherein the air flow control device is configured to automatically release from the second coupler and expand from the collapsed configuration into the expanded configuration after the air flow control device is pushed out of the delivery sheath.

24. The assembly of an air flow control device and an insertion tool of claim 23 further comprising a mandrel wire within the delivery shaft, wherein the mandrel wire is configured to extend from the distal end of the delivery shaft and function to secure engagement of the first and second couplers.

25. The flow control device of claim 1, wherein the wall has an outer perimeter aligned with a distal end of the hollow structural frame.

26. The flow control device of claim 1, wherein the structural frame does not extend distally of the wall.

27. The flow control device of claim 1, wherein the one-way valve is included in the sealing membrane.

28. The flow control device of claim 12, wherein the one-way valve is entirely distal of the spokes.
